(19) United States
(12) Patent Application Publication
(10) Pub. No.: US 2026/0070103 A1
(43) Pub. Date: Mar. 12, 2026

(54) MITIGATION OF PERSISTENT HARMFUL COMPOUNDS

(71) Applicant: Worcester Polytechnic Institute, Worcester, MA (US)

(72) Inventors: Suzanne F. Scarlata, Worcester, MA (US); Maxwell Markey, Worcester, MA (US); Carissa Olsen, Shrewsbury, MA (US); Madison Rennie, Fiskdale, MA (US); Mark Xatse, Worcester, MA (US)

(21) Appl. No.: 19/323,844

(22) Filed: Sep. 9, 2025

Related U.S. Application Data
(60) Provisional application No. 63/692,925, filed on Sep. 10, 2024.

(51) Int. Cl. B09C 1/10; C12N 9/14; C12N 15/78; C12N 15/85

(52) U.S. Cl. CPC B09C 1/10; C12N 9/14; C12N 15/78; C12N 15/8509; C12N 2800/101; C12N 2800/103; C12Y 308/01002; C12Y 308/01003

(57) ABSTRACT

A method of mitigating and rehabilitating soil and land areas contaminated with forever chemicals such as PFAS (perfluoroalkyl and polyfluoroalkyl) based toxins involves manipulating genetic nucleotides of carrier organisms for expressing an enzyme reactive with the forever chemicals for rendering them benign. The carrier organisms pass the modified DNA that enables the enzyme to subsequent generations for maintaining an active population of enzyme-expressing organisms, typically worms, once introduced.

### RELATED APPLICATIONS

This patent application claims the benefit under 35 U.S.C. § 119(e) of U.S. Provisional Patent App. No. 63/692,925, filed Sep. 10, 2024, entitled “MITIGATION OF PERSISTENT HARMFUL COMPOUNDS,” incorporated herein by reference in entirety.

### BACKGROUND

So called “forever chemicals” such as perfluorooctane, sulfonic acid and related compounds remain in the environment for an extended period, without degrading, posing long-term health risks. Available approaches to degrade these compounds are typically chemical methods and are not cost-effective. Substantial cost, time and effort are associated with remediation of land areas and soil contaminated with forever chemicals. In particular, PFAS, referring to perfluoroalkyl and polyfluoroalkyl substances, are synthetic chemicals whose strong carbon-fluorine bonds make them extremely resistant to degradation. Therefore, they tend to persist in the environment and the human body for an extended period of time.

### SUMMARY

A method of mitigating and rehabilitating soil and land areas contaminated with forever chemicals involves manipulating genetic nucleotides of carrier organisms for expressing an enzyme reactive with the forever chemicals to render them benign. The carrier organisms pass the modified DNA that enables the enzyme to subsequent generations for maintaining an active population of enzyme-expressing organisms, typically worms or bacteria, once introduced.

The disclosed approach to eradication of environmental toxins includes modifying a carrier organism to express an active enzyme capable of breaking down soil-bound toxins, and introducing the carrier organism to a land region afflicted with the toxins. Once introduced, the carrier organism expresses the active enzyme to soil in the land region. This may be preceded by injecting the carrier organism with a nucleotide including the active enzyme, and subsequently expressing the active enzyme via the carrier organism. Ordinary reproductive activity then propagates generic material including DNA for expressing the active enzyme to successive generations of the carrier organism.

Configurations herein are based, in part, on the observation that highly stable compounds such as the PFAS substances and others can persist for an extended period without degrading or reacting with naturally occurring materials to achieve a benign state. Unfortunately, conventional approaches to remediation of contaminated areas suffer from the shortcoming of high costs, high heat and/or labor intensive approaches that may not be feasible. Accordingly, configurations herein substantially overcome the shortcoming of conventional toxicity mitigation by providing a nucleic sequence vector for modifying DNA or RNA of an organism for expressing an enzyme that breaks down or otherwise renders harmless toxic substances such as PFAS materials.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a PFOS mitigation environment responsive to configurations herein;

FIG. 2 is a schematic diagram of a toxic substance mitigation and degradation approach in the environment of FIG. 1;

FIG. 3 shows a flowchart of a toxic substance mitigation and degradation process in the environment of FIGS. 1 and 2; and

FIG. 4 is a process flow of a particular configuration for mitigating PFOS.

### DETAILED DESCRIPTION

A method of mitigating environmental toxins includes modifying a carrier organism for populating a contaminated region such as an earthen area. The modified carrier organism is preferably a worm or bacteria amenable to soil propagation, although any suitable organism responsive to the enzyme inducing nucleic sequence vector may benefit from the disclosed approach.

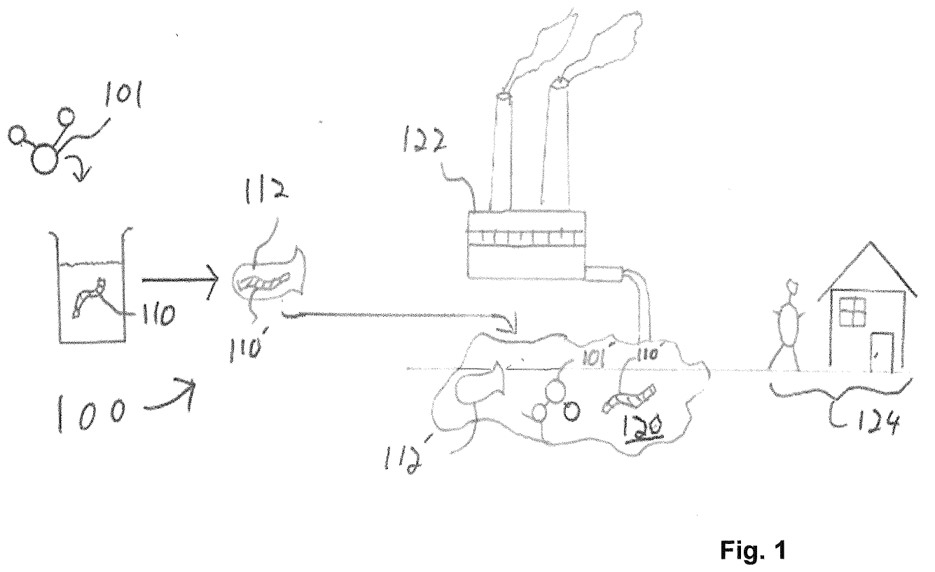

FIG. 1 is a context diagram of a PFAS mitigation environment 100 responsive to configurations herein. Referring to FIG. 1, a method for mitigating and degrading a toxic substance such as PFAS compounds includes identifying an enzyme 101 having an affinity for a target substance such as PFAS, and forming a nucleic sequence vector 110 including the identified enzyme. The nucleic sequence vector 110′ is introduced into an organism 112, and the organism 112 deployed in or around a toxic remediation site 120 such as one subject to industrial waste or activity 122. PFAS compounds, particularly in a discarded soil/earthen area, can be particularly harmful and elusive to occupants and residences 124 around the site 120. Migrated organisms 112′ then express the enzyme 101′ in the presence of the toxic substance via replication of a nucleic acid sequence including the nucleic sequence vector 110′ in the organism.

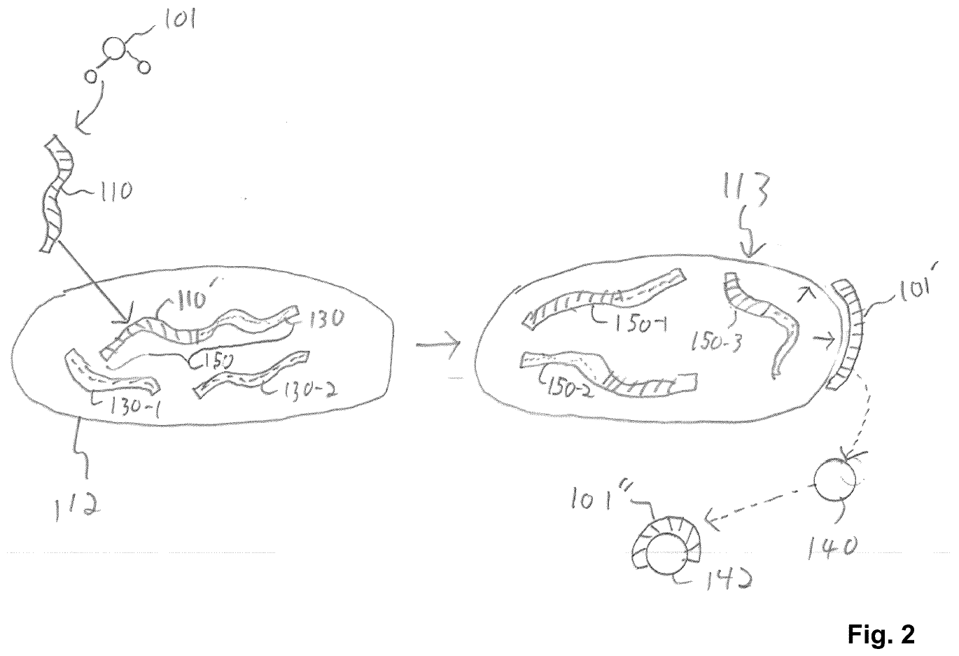

FIG. 2 is a schematic diagram of a toxic substance mitigation and degradation approach in the environment of FIG. 1. Referring to FIGS. 1 and 2, deployment in the site 120 commences with forming a plasmid or peptide defining the nucleic sequence vector 110. A bioinformatic or genetic modification approach appends the plasmid or peptide to a nucleic sequence 130 native to the organism 112. The organism 112 with the now modified nucleic acid sequence 150-1 . . . 150-3 (150 generally), typically a modified strand of DNA or RNA, is now capable if expressing the enzyme 101′ via the nucleic sequence vector 110 on a surface 113 of the organism 112 based on replication of the modified nucleic acid sequence 150 including the nucleic sequence vector 110, typically a plasmid capable of expressing or generating the enzyme 101 . . . . By combining the nucleic sequence vector 110 with a natural DNA or RNA strand 130 in the organism, the enzyme 101 is then expressed to the surface 113 of the organism based on the DNA or RNA replication in the organism. The enzyme 101″ expressed based on the nucleic sequence vector 110′ is in communication with the soil having PFAS 140 molecules and combines, degrades or otherwise forms a mitigated PFAS compound 142 in conjunction with the deployed enzyme 101″ which is now continuously introduced to the surface 113 of the organism 112 through replication of the modified DNA/RNA strand 130.

In terms of cell biochemistry, in an example configuration, the nucleic sequence vector 110 contains the DNA for the enzyme 101 along with ‘instructions’ on where in the organism's 112 cell the enzyme 101 should be localized. The vector DNA will be transcribed into RNA and the protein will be made and transported to the precise location in the cell. These processes are part of the normal cell machinery.

In the example of FIG. 2, protein expression in bacteria is like turning bacteria into tiny “factories” for ongoing metabolic generation of DeHa2 as the expressed enzyme 101′. A particular configuration provides the bacteria with a “recipe” for the protein (DeHa2) by inserting a piece of DNA (a plasmid) carrying the instructions in the vector 110. The bacteria organism 112 reads this recipe just like they do with their own DNA, and as they grow and multiply, they start making large amounts of the protein (DeHa2). Since bacteria reproduce very quickly, millions of cells can concurrently generate the protein (DeHa2) at once via the modified nucleic acid sequence 150, and expressing the enzyme 101′ to the surrounding soil 120.

An alternate configuration may employ the organism 112 for collecting the bacteria, break them open, and purify the protein so it can be harvested for medicine, research, or industry. The configuration of FIG. 2 depicts expressing the enzyme in and on the surface 113 of the cell.

Configurations herein employ organisms suited for soil propagation for targeting PFAS toxins, however the enzyme expression induced by generic modification may also be applied to other contaminants and organisms. The examples herein employ PFAS as a target substance, enumerated in Table I below:

TABLE I

| PFAS | | | |
|---|---|---|---|
| Type | Full Name | Common Uses | Health Concerns |
| PFOA | Perfluorooctanoic | Nonstick | Linked to cancer, liver |
| | acid | cookware, | damage |
| | | waterproof | |
| | | fabrics | |
| PFOS | Perfluorooctane | Firefighting | Thyroid disease, |
| | sulfonate | foam, stain | developmental issues |
| | | repellents | |
| GenX | HFPO-DA | Industrial | Emerging concerns, less |
| | (replacement | manufacturing | studied |
| | for PFOA) | | |
| PFNA | Perfluorononanoic | Electronics, | Reproductive and |
| | acid | coatings | immune effects |
| PFHxS | Perfluorohexane | Textiles, | Bioaccumulates in blood, |
| | sulfonate | firefighting | liver toxicity |
| | | foam | |

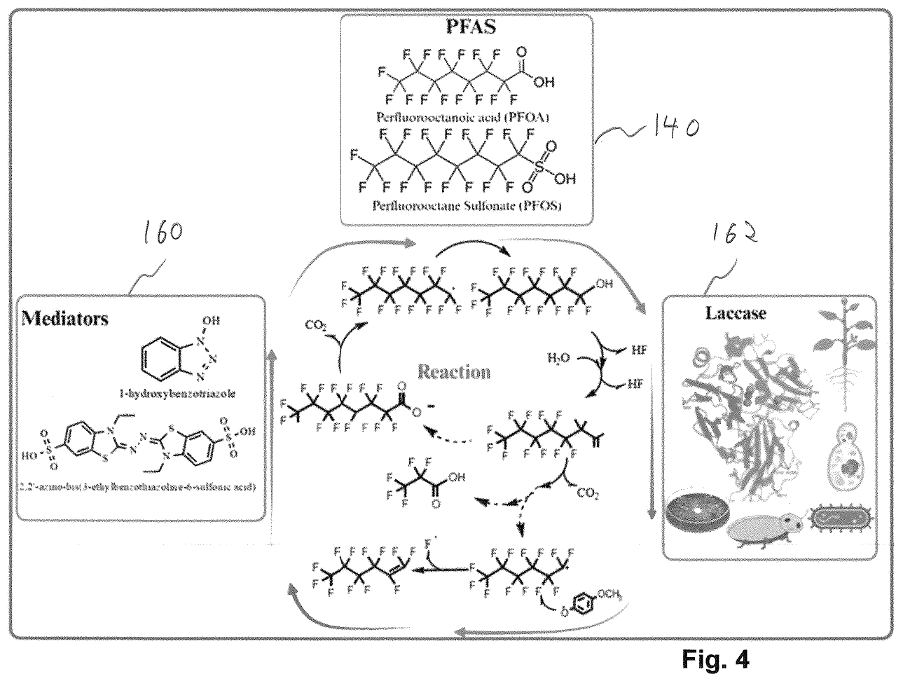

Because of the density of the electron clouds of the fluorinated groups, PFAS compounds are very difficult to access chemically. Conventional approaches include the use of high temperatures and pressures, electrochemical oxidation, plasma-based treatment to generate reactive radicals. These processes are not economical or energy efficient. Configurations disclosed herein describe biological methods to degrade PFAS that incorporate enzymes with the aim of soil remediation. These methods are both based on enzymes that degrade PFAS. In one configuration, these enzymes are expressed in worms that are self-propagating and can be placed in the soil for remediation. Worms allow an inexpensive renewable resource since they lay ˜300 eggs during their lifetime. In a second application, these enzymes are expressed on the surface of a bacteria (Pseudomonas) that adheres to the roots of plants. Seedlings can be inoculated with the bacteria and planted so that remediation occurs in the surrounding soil and the food product is safe. In another application, configurations express DeHa1 on the surface of bacteria that adhere to the roots of plants, discussed in FIG. 4 below. The preferred configuration seeks to develop robust “forever chemical” digesting organisms that digest these toxic target substances into safe and non-toxic molecules.

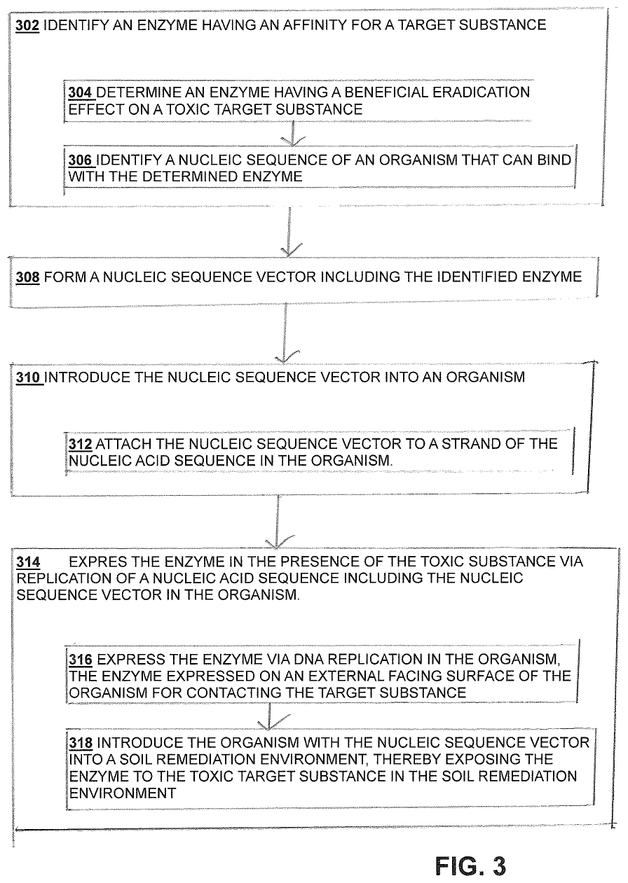

FIG. 3 shows a flowchart of a toxic substance mitigation and degradation process in the environment of FIGS. 1 and 2. In molecular modification of genetic material, a vector is any particle (e.g., plasmids, cosmids, Lambda phages) used as a vehicle to artificially carry a foreign nucleic sequence—usually DNA—into another cell, where it can be replicated and/or expressed A vector containing foreign DNA is termed recombinant DNA. The four major types of vectors are plasmids, viral vectors, cosmids, and artificial chromosomes. A peptide is a short chain of amino acids, while a plasmid is a circular piece of double-stranded DNA that naturally occurs in bacteria and is used as a vector in genetic engineering to carry and replicate foreign genes into cells. Of these, the most commonly used vectors are plasmids. Common to all engineered vectors are the origin of replication, a multicloning site, and a selectable marker.

The vector itself generally carries a DNA sequence that consists of an insert (in this case the transgene) and a larger sequence that serves as the “backbone” of the vector. The purpose of a vector which transfers genetic information to another cell is typically to isolate, multiply, or express the insert in the target cell. All vectors may be used for cloning and are therefore cloning vectors, but there are also vectors designed specifically for cloning, while others may be designed specifically for other purposes, such as transcription and protein expression. Vectors designed specifically for the expression of the transgene in the target cell are called expression vectors, and generally have a promoter sequence that drives the expression of the transgene. Simpler vectors called transcription vectors are only capable of being transcribed but not translated: they can be replicated in a target cell but not expressed, unlike expression vectors. Transcription vectors are used to amplify their insert.

Referring to FIGS. 1-3, the method for mitigating and degrading a toxic substance as disclosed herein includes, at step 302, identifying an enzyme having an affinity for a target substance. The affinity refers to an ability and/or tendency to molecularly combine with molecules of the target substance such as PFAS 140, and combine, react, surround or otherwise render the target substance harmless. This includes determining an enzyme 101 having a beneficial eradication effect on the toxic target substance, as depicted at step 304, and identifying a nucleic sequence (typically DNA or RNA) of an organism 112 that can bind with a plasmid for generating the determined enzyme 101, depicted at step 306.

A nucleic sequence vector 110 is then formed including DNA or “instructions” for expressing or generating the identified enzyme 101, as shown at step 308. The net result is that the sequence vector 110 either includes the enzyme 101 and/or enables the organism 112 to express the enzyme 101′. The nucleic sequence vector is then injected into the organism 112, as depicted at step 310. Introducing the nucleic sequence vector 100 further includes attaching the nucleic sequence vector to a strand of the nucleic acid sequence in the organism, where it effectively becomes part of the organism 112 physiology and is replicated as part of the modified nucleic acid sequence 150, or DNA strand, with which it is associated/attached, as shown at step 312.

The modified organism is now adapted for expressing the enzyme 101 in the presence of the toxic substance via replication of the nucleic acid sequence 150 including the nucleic sequence vector 110 in the organism 112, as depicted at step 314. Subsequently, the organism 112 expresses the enzyme 101 via DNA replication in the organism, such that the enzyme is expressed on an external facing surface 113 of the organism for contacting the target substance 140, as depicted at step 316. The organism 112 is then introduced into a soil remediation environment, thereby exposing the enzyme 101′ to the toxic target substance 140 in the soil remediation environment 100, as shown at step 318.

In the example of FIG. 1, the organism is selected based on a compatibility with an earthen environment for mitigating a soil site 120. The examples above are particularly well suited when the target substance is a fluorine compound, such as perfluoroalkyl and polyfluoroalkyl (PFAS) substances. Deployment in a soil site 120 is well suited to the organism Caenorhabditis elegans (C. elegans), which can include colonizing a plurality of C. elegans specimens in a contaminated soil region, as the C. elegans are wormlike organisms suited for soil habitation. Alternatively, the organism may be Pseudomonas bacteria amenable to plant roots, including inoculating seedlings of a plant species with the bacteria, and planting the seedlings in a contaminated soil region, thereby inducing remediation in a soil region surrounding a root structure of the plant species. By introducing the root structure into a contaminated soil region, the root-adhered bacteria are encouraged to express the enzyme 101.

FIG. 4 is a process flow of a particular configuration for mitigating PFAS using DeHa2 as the enzyme 101. Referring to FIGS. 1-4, an enzyme DeHa2 has been established to have activity towards PFAS compounds. Approaches known as directed evolution may be invoked to accelerate natural evolution in a much quicker and controlled manner to develop enzymes with enhanced activity. A combination of site saturation and a mutator strain E. coli XL1Red cells is employed to optimize and improve the enzymatic activity of DeHa2. Bioinformatic approaches assist with identifying residues of interest on DeHa2 that are present around the punitive active site and other identified site residues are known for high activity. The identified sites will be mutated using 5Q site-directed mutagenesis to generate a diverse library. The DeHa2 pET-52(b) plasmid will be transformed into Bl21 (expression cells), E. coli XL1-red (mutator cells), and 10-beta (stable cells). The resulting enzymes will then be assayed for catabolic PFAS activity, using LC-MS based techniques.

In an alternate approach, DeHa2 couples with a second enzyme system (Laccase/mediator) to more fully degrade PFAS and express these together in a coupled system in Pseudomonas. As we design better enzyme variants using computational methods and directed evolution techniques, variants may be tested by first synthesizing the DNA, inserting the DNA into a carrier that will allow the bacteria or the worms to convert the DNA into RNA and then synthesize the enzyme to a protein that sits on the outer surface of the bacteria.

Mediators 160 facilitate combination with PFAS molecules 140, while the Lacasse reactions 162 strip some of the fluorine (HF) in a cyclic manner. Laccases, refers to broad spectrum biocatalysts that are employed to degrade several compounds, such as those that can be found in the effluents from industries and hospitals. Laccases have shown high potential in the biotransformation of diverse pollutants using crude enzyme extracts or free enzymes.

The implementation in a contaminated soil region is particularly amenable to the Caenorhabditis elegans organism 112 because it can be freely deployed to thrive in the soil environment. C. elegans is a free-living transparent nematode about 1 mm in length. Transgenic C. elegans can be readily created via microinjection of a DNA plasmid solution into the gonad. The plasmid DNA rearranges to form extrachromosomal concatemers that are stably inherited, though not with the same efficiency as actual chromosomes. A gene of interest is co-injected with an obvious phenotypic marker, such as rol-6 or GFP, to allow selection of transgenic animals under a dissecting microscope. The exogenous gene may be expressed from its native promoter for cellular localization studies. Alternatively, the transgene can be driven by a different tissue-specific promoter to assess the role of the gene product in that particular cell or tissue. This technique efficiently drives gene expression in all tissues of C. elegans except for the germline or early embryo. Creation of transgenic animals is widely utilized for a range of experimental paradigms. A microinjection procedure may be employed to generate transgenic worms. Furthermore, selection and maintenance of stable transgenic C. elegans lines supports a continuing population of C. elegans worms for expressing the mitigating enzyme.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for mitigating and degrading a toxic substance, comprising:
identifying an enzyme having an affinity for a target substance;
forming a nucleic sequence vector associated with the identified enzyme;
introducing the nucleic sequence vector into an organism; and
expressing the enzyme in the presence of the toxic substance via replication of a nucleic acid sequence including the nucleic sequence vector in the organism.

2. The method of claim 1 wherein identifying the enzyme further comprises:
determining an enzyme having a beneficial eradication effect on a toxic target substance;
identifying a nucleic sequence of an organism that can bind with the determined enzyme.

3. The method of claim 1 wherein introducing the nucleic sequence vector further comprises attaching the nucleic sequence vector to a strand of the nucleic acid sequence in the organism.

4. The method of claim 1 further comprising:
expressing the enzyme via DNA replication in the organism, the enzyme expressed on an external facing surface of the organism for contacting the target substance.

5. The method of claim 1 further comprising:
generating a plasmid having a capability for generating the enzyme;
forming the nucleic sequence vector including the plasmid.

6. The method of claim 5 wherein the plasmid is a DNA strand for inducing the enzyme, further comprising:
appending the plasmid to the nucleic acid sequence in the organism; and
transcribing the plasmid to RNA for replication and generation of the enzyme.

7. The method of claim 6 wherein the enzyme includes a DeHa2 protein.

8. The method of claim 1 further comprising
selecting the organism based on a compatibility with an earthen environment; and
introducing the organism with the nucleic sequence vector into a soil remediation environment, thereby exposing the enzyme to the toxic target substance in the soil remediation environment.

9. The method of claim 1, further comprising:
forming a plasmid or peptide defining the nucleic sequence vector;
appending the plasmid or peptide to a nucleic sequence native to the organism; and
expressing the enzyme on a surface of the organism based on replication of the nucleic sequence including the nucleic sequence vector.

10. The method of claim 1 further comprising:
combining the nucleic sequence vector with a natural DNA or RNA strand in the organism, the enzyme expressed to a surface of the organism based on DNA or RNA replication.

11. The method of claim 1 wherein the organism is Caenorhabditis elegans (C. elegans), further comprising colonizing a plurality of C. elegans specimens in a contaminated soil region.

12. The method of claim 1 wherein the organism is Pseudomonas bacteria, further comprising:
inoculating seedlings of a plant species with the bacteria; and
planting the seedlings in a contaminated soil region, thereby inducing remediation in a soil region surrounding a root structure of the plant species.

13. The method of claim 1 wherein the organism is Pseudomonas bacteria, further comprising: adhering the organism to a root structure of a plant; and
introducing the root structure into a contaminated soil region, thereby encouraging root-adhered bacteria to express the enzyme.

14. The method of claim 1 wherein the target substance is a fluorine compound.

15. The method of claim 12 wherein the target substance includes perfluoroalkyl and polyfluoroalkyl (PFAS) substances.

16. A system for mitigating and degrading a toxic substance, comprising:
an enzyme having an affinity for a target substance;
a nucleic sequence vector associated with the identified enzyme;
an injection mechanism for introducing the nucleic sequence vector into an organism, the organism adapted for expressing the enzyme in the presence of the toxic substance via replication of a nucleic acid sequence including the nucleic sequence vector in the organism.

17. The system of claim 16 further comprising:
a genetic manipulation process for combining the nucleic sequence vector to a strand of the nucleic acid sequence in the organism.
